(19) United States
(12) Patent Application Publication
(10) Pub. No.: US 2019/0022270 A1
(43) Pub. Date: Jan. 24, 2019

(54) FANLESS NEGATIVE IONIC WIND AROMA DIFFUSER

(71) Applicant: KUO-FU YANG, TAIPEI CITY (TW)

(72) Inventor: KUO-FU YANG, TAIPEI CITY (TW)

(21) Appl. No.: 15/657,232

(22) Filed: Jul. 24, 2017

(51) Int. Cl. A61L 9/22; A61L 9/03; H01T 23/00

(52) U.S. Cl. CPC A61L 9/22; A61L 9/03; A61L 2209/13; A61L 2209/11; H01T 23/00

(57) ABSTRACT

A fanless negative ionic wind aroma diffuser includes a housing, a control circuit board, an essential oil container, a heating member, and a negative ion generation module. The control circuit board, the essential oil container, the heating member, and the negative ion generation module are mounted in an accommodation space inside the housing. The control circuit board is electrically connected with an external power source for supplying the required power to the heating member and the negative ion generation module. When actuated, the negative ion generation module generates a negative ionic wind for blowing out fragrant particles produced by heating an essential oil in the essential oil container to be quickly diffused around the surrounding, without a fan. In addition, negative ions are beneficial to health.

### FIELD OF INVENTION

The present invention relates to an aroma generation device, and more particularly to a fanless negative ionic wind aroma diffuser which is provided with a negative ion generation module to generate a negative ionic wind when actuated for diffusing fragrant particles generated by heating an essential oil to attain aromatherapy.

### BACKGROUND OF INVENTION

### 1. Description of the Related Art

Essential oils are derived from natural flowers, leaves, peel or bark, and the like by extracting to get volatile plant oils. The specific aromatic scents of plants can be used for aromatherapy. Users utilize blends of therapeutic essential oils that can be issued through topical application, massage, inhalation or water immersion to stimulate a desired response for improving psychological or physical well-being. In general, an aroma diffuser is placed in a room, car, office etc., to improve air quality and to refresh the user. Most of aroma diffusers produce aromas by self-evaporation, burning, heating or high-frequency vibration, etc. It is quite difficult for essential oils to be diffused around the surrounding in a short time by the evaporation of the essential oil itself. Therefore, a fan is used to diffuse the aroma after evaporated into the surrounding. This design is also applied to an aroma diffuser which is implemented by burning, heating or high-frequency vibration. However, there is a noise generated when the fan is running, and whether the aroma can be blown out is also a big problem. Especially, if the evaporation is not complete or the heating effect is poor, the large aromatic particles will be blown out to cause the problem of a thick aroma and the user may feel uncomfortable.

In view of this, the inventor of the present invention has devoted himself based on his many years of practical experiences to solve these problems and develop a fanless negative ionic wind aroma diffuser which is able to generate a negative ionic wind to diffuse fragrant particles produced by heating an essential oil, without a fan.

### 2. Summary of the Invention

The primary object of the present invention is to provide a fanless negative ionic wind aroma diffuser which is reasonable in structure and is easy to use. The fanless negative ionic wind aroma diffuser comprises a housing. The housing is provided with an essential oil container, a heating member, and a negative ion generation module therein. The negative ion generation module is disposed close to an opening of the housing to generate a negative ionic wind for blowing out fragrant particles produced by heating an essential oil in the essential oil container to be quickly diffused around the surrounding, without a fan. In addition, negative ions are beneficial to health.

In order to achieve the aforesaid object, the fanless negative ionic wind aroma diffuser of the present invention comprises a housing, a control circuit board, an essential oil container, a heating member, and a negative ion generation module. The housing has an accommodation space therein. A top of the housing is provided a grille-like opening. The control circuit board is disposed in the accommodation space and electrically connected with an external power source. The essential oil container is disposed in the accommodation space. The essential oil container is used for containing an essential oil. The heating member is disposed in the accommodation space and electrically connected with the control circuit board. The heating member is located under a bottom of the essential oil container to heat the essential oil for producing a fragrant particle. The negative ion generation module is disposed in the accommodation space and electrically connected with the control circuit board. The negative ion generation module is located corresponding to one side of the grille-like opening. When the negative ion generation module is actuated, the negative ion generation module generates a negative ionic wind to bring the fragrant particle out from the grille-like opening.

In an embodiment, the housing includes a pair of a first housing and a second housing. The first housing is provided with a plurality of through holes to form the grille-like opening. One side of the second housing is provided with an opening corresponding to the essential oil container. The essential oil container is made of a heat conductive material. An upper opening of the essential oil container is sealed with a waterproof breathable film. Furthermore, the fanless negative ionic wind aroma diffuser further comprises a plurality of negative ion emitting needles disposed inside the first housing and corresponding in position to the through holes. The negative ion emitting needles are electrically connected with negative ion generation module. It is to be noted that the negative ion emitting needles are arranged in pairs, for example, the number of the negative ion emitting needles may be two, four or six. Preferably, the number of the negative ion emitting needles is four, and the negative ion emitting needles are spaced apart from each other. The heating member is a heat generation plate, and the heat generation sheet is formed with a fence-shaped slot corresponding to the essential oil container for creating a greater heating area to speed up the evaporation of the essential oil.

Besides, the control circuit board is provided with a switch and a socket on a surface of the housing. The control circuit board includes a timer thereon for respectively controlling the heating member and the negative ion generation module to actuate and bring a time difference therebetween to fully diffuse aroma.

### BRIEF DESCRIPTION OF THE DRAWINGS

FIG. 1 is an exploded view in accordance with a preferred embodiment of the present invention;

FIG. 2 is a sectional view in accordance with the preferred embodiment of the present invention after assembled;

FIG. 3 is a schematic view showing the configuration of the preferred embodiment of the present invention after assembled; and

FIG. 4 is a sectional view in accordance with the preferred embodiment of the present invention when in use.

### DESCRIPTION OF THE PREFERRED EMBODIMENTS

The above and other objects, features and advantages of this disclosure will become apparent from the following detailed description taken with the accompanying drawings.

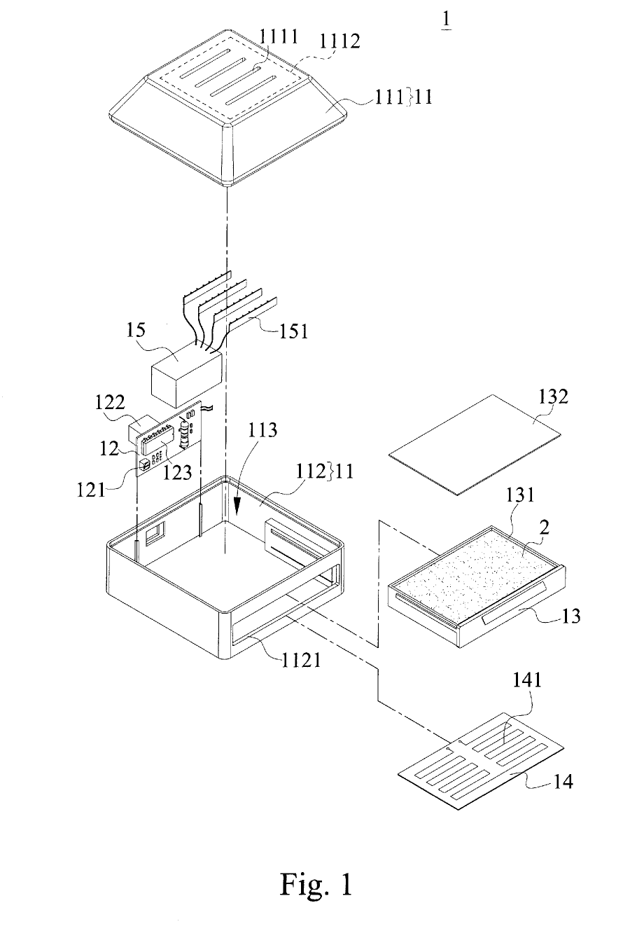

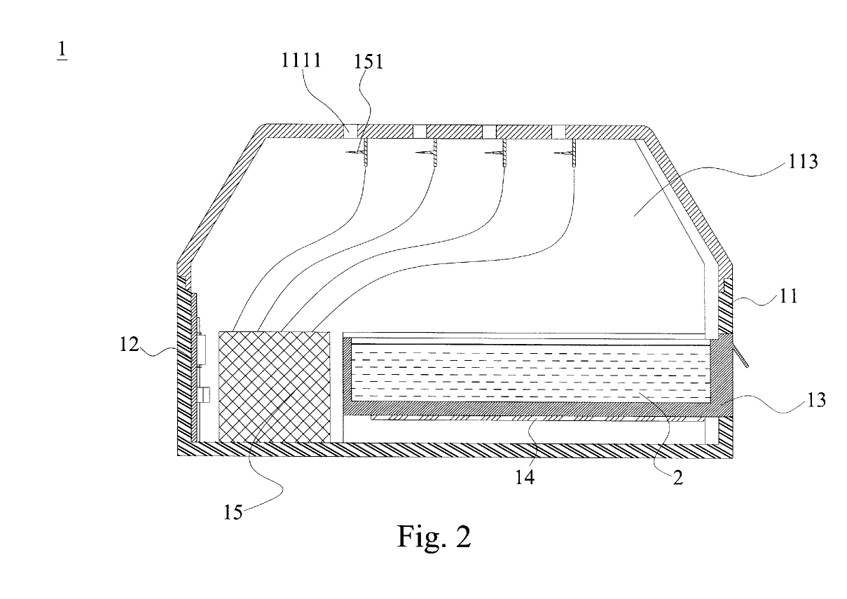

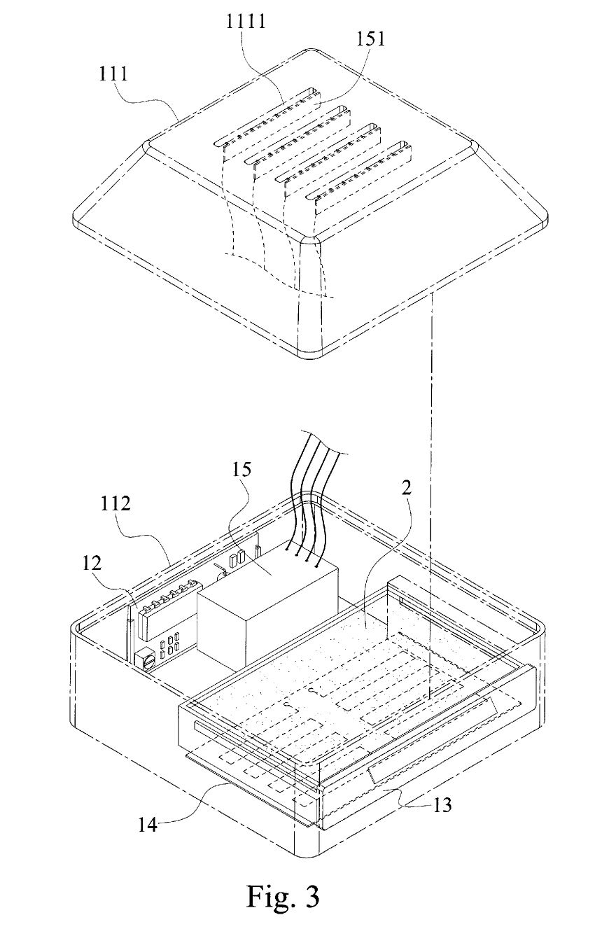

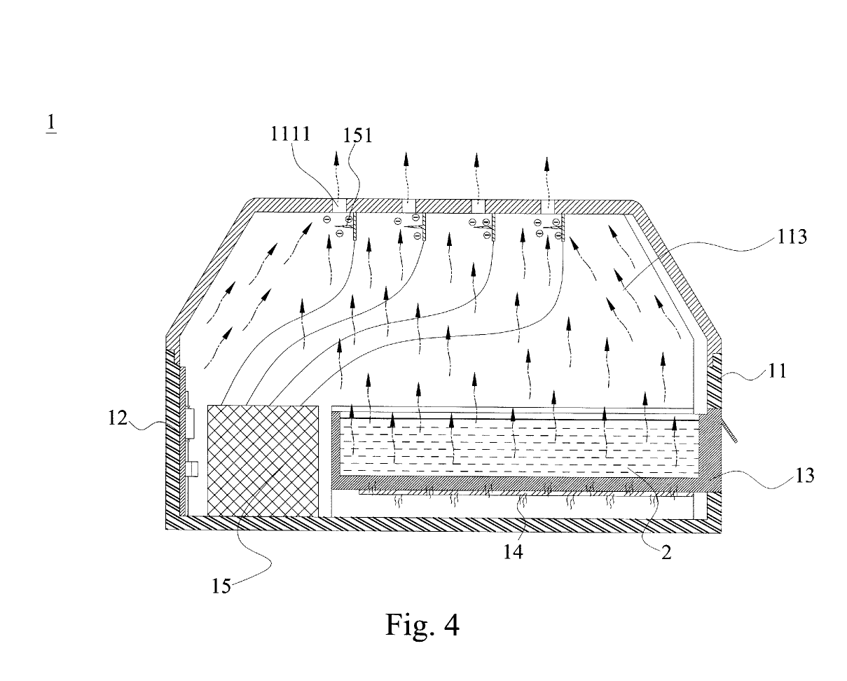

FIG. 1 is an exploded view in accordance with a preferred embodiment of the present invention. FIG. 2 is a sectional view in accordance with the preferred embodiment of the present invention after assembled. FIG. 3 is a schematic view showing the configuration of the preferred embodiment of the present invention after assembled. FIG. 4 is a sectional view in accordance with the preferred embodiment of the present invention when in use. As shown in the drawings, a fanless negative ionic wind aroma diffuser of the present invention comprises a housing 11, a control circuit board 12, an essential oil container 13, a heating member 14, and a negative ion generation module 15.

The housing 11 includes a pair of a first housing 111 and a second housing 112 to form an accommodation space 113 therein. A top of the first housing 111 is provided with a plurality of through holes 1111 to form a grille-like opening 1112. One side of the second housing 112 is provided with an opening 1121 corresponding to the essential oil container 13.

The control circuit board 12 is disposed in the accommodation space 113. The control circuit board 12 is provided with a switch 121 and a socket 122 on the surface of the housing 11. The control circuit board 12 includes a timer 123 thereon. Through the socket 122, the control circuit board 12 is electrically connected with an external power source (not shown). The switch 121 is used to turn on/off the power supply as the main means to actuate the present invention.

The essential oil container 13 is a rectangular container, and is made of a heat conductive material. An upper portion of the essential oil container 13 has an upper opening 131 for containing an essential oil 2. The upper opening 131 is sealed with a waterproof breathable film 132, so that the essential oil 2 in the essential oil container 13 is not spilled when used. The essential oil container 13 is inserted into the accommodation space 113 from the opening 121 in the manner of inserting a card.

The heating member 14 is a heat generation plate. The heat generation sheet is formed with a fence-shaped slot 141 corresponding to the bottom of the essential oil container 13. The heating member 14 is disposed in the accommodation space 113 and electrically connected with the control circuit board 12. The heating member 14 is located under the bottom of the essential oil container 13 to heat the essential oil 2 for producing fragrant particles.

The negative ion generation module 15 is disposed in the accommodation space 113 and electrically connected with the control circuit board 12. The negative ion generation module 15 includes a plurality of negative ion emitting needles 151 which are spaced apart from each other and arranged in pairs in the through holes 1111 of the grille-like opening 1112. The number of the negative ion emitting needles 151 may be two, four or six. After actual tests, the number of the negative ion emitting needles 151 is preferably four.

In the vicinity of the negative ion emitting needles 151, since the local electric field intensity exceeds the electric field strength of the gas, enabling the gas to be ionized and excited, so that a relatively stable corona discharge occurs, and the positive ions flow to the negative electrode to meet the negative ions, so it can facilitate the air flow to form a relatively stable air flow, known as a negative ionic wind, so that the fragrant particles can be sent out from the grille-like opening 1112. In addition, the timer 123 is electrically connected with the heating member 14 and the negative ion generation module 15 for respectively controlling the heating member 14 and the negative ion generation module 15 to generate a time difference therebetween. After the essential oil 2 is sufficiently heated by the heating member 14, the aroma of the fragrant particles is dispersed by the negative ionic wind.

In summary, compared to the conventional aromatic essential oil box, the fanless negative ionic wind aroma diffuser of the present invention needn't use a fan to blow the fragrant particles produced by heating the essential oil, so that there is no noise generated. The fragrant particles of the essential oil, blown by the negative ionic wind, are not only environmentally friendly but also diffused at a high speed. In addition, the negative ions generated by the negative ion generation module 15 have the advantage of improving lung function, promoting metabolism, enhancing a resistance to diseases, improving sleep, removing dust and exterminating bacteria, cleaning the air, and so on.

While the invention has been described by means of specific embodiments, numerous modifications and variations could be made thereto by those skilled in the art without departing from the scope and spirit of the invention set forth in the claims.

What is claimed is:

1. A fanless negative ionic wind aroma diffuser, comprising:
a housing, having an accommodation space therein, a top of the housing being provided a grille-like opening;
a control circuit board, disposed in the accommodation space and electrically connected with an external power source;
an essential oil container, disposed in the accommodation space, the essential oil container being used for containing an essential oil;
a heating member, disposed in the accommodation space and electrically connected with the control circuit board, the heating member being located under a bottom of the essential oil container to heat the essential oil for producing a fragrant particle; and
a negative ion generation module, disposed in the accommodation space and electrically connected with the control circuit board, the negative ion generation module being located corresponding to one side of the grille-like opening, wherein when the negative ion generation module is actuated, the negative ion generation module generates a negative ionic wind to bring the fragrant particle out from the grille-like opening.

2. The fanless negative ionic wind aroma diffuser as claimed in claim 1, wherein the housing includes a pair of a first housing and a second housing, the first housing is provided with a plurality of through holes to form the grille-like opening, and one side of the second housing is provided with an opening corresponding to the essential oil container.

3. The fanless negative ionic wind aroma diffuser as claimed in claim 2, wherein the essential oil container is made of a heat conductive material.

4. The fanless negative ionic wind aroma diffuser as claimed in claim 3, wherein an upper opening of the essential oil container is sealed with a waterproof breathable film.

5. The fanless negative ionic wind aroma diffuser as claimed in claim 4, further comprising a plurality of negative ion emitting needles disposed inside the first housing and corresponding in position to the through holes, the negative ion emitting needles being electrically connected with negative ion generation module.

6. The fanless negative ionic wind aroma diffuser as claimed in claim 5, wherein the heating member is a heat generation plate, and the heat generation sheet is formed with a fence-shaped slot.

7. The fanless negative ionic wind aroma diffuser as claimed in claim 6, wherein the control circuit board is provided with a switch and a socket on a surface of the housing, the control circuit board includes a timer thereon for respectively controlling the heating member and the negative ion generation module to actuate and bring a time difference therebetween to fully diffuse aroma.
